(19) United States
(12) Patent Application Publication
(10) Pub. No.: US 2008/0147047 A1
(43) Pub. Date: Jun. 19, 2008

(54) Needleless Hub Disinfection Device and Method

(75) Inventors: Gregory T. Davis, Crystal Lake, IL (US); Lawrence G. Ponsi, Wheeling, IL (US); Jeffrey B. Steffens, Cary, IL (US); Paul H. Hanifl, Barrington Hills, IL (US)

Correspondence Address: BARNES & THORNBURG LLP, P.O. BOX 2786, CHICAGO, IL, 60690-2786, US

(73) Assignee: SAGE PRODUCTS, INC., Cary, IL (US)

(21) Appl. No.: 11/611,927

(22) Filed: Dec. 18, 2006

(51) Int. Cl. A61M 39/16

(52) U.S. Cl. 604/533

(57) ABSTRACT

A device for disinfecting and protection a portion of a medical implement, such as a needleless hub or injection port. The device includes a shroud in the form of a cap which is shaped to engage the medical implement, and a disinfectant pad located in the cap engages the needleless hub or injection port, when the cap is applied, to disinfect and protect the hub or port. A covering is provided for maintaining liquid disinfectant in the pad to prevent loss or evaporation.

### BACKGROUND OF THE INVENTION

This invention related to medical implements such as needleless hubs or injection ports, and in particular to a device for disinfecting the hub or port and, when applied, keeping the hub or port covered, cleaned and disinfected.

Needleless vascular catheter hubs and access or injection ports are used thousands of times each day in the United States medical facilities. Unless the hubs and ports are disinfected, patients are at a significant risk of blood stream infections caused by microbes that gain access through the needleless hub or injection port.

In the past, practitioners using a needleless hub or injection port have sought to disinfect the hub or port with alcohol in order to prevent microbial entry. Practitioners who seek disinfection in this manner typically wipe the hub or injection port with an alcohol-soaked swab before accessing it. That, however, has proven to be only partially successful in reducing blood stream infections which are introduced through the needleless hubs or injection ports.

U.S. Pat. Nos. 5,554,135, 5,792,120 and 6,045,539 deal with the infection problem by providing a cover for the injection port, with the cover including a sponge and shatterable plastic capsule containing an antiseptic solution. When the cover is applied to a needleless hub or injection port and the plastic capsule is shattered, disinfectant soaks the sponge and disinfects the covered end of the needleless hub or injection port.

One form of a needleless hub disinfection device is disclosed in copending U.S. patent application Ser. No. 11/132,902, the disclosure of which is incorporated herein by reference and which is owned by the owner of the present application. The present application, albeit more manual in use than the incorporated application, provides a simple and effective means of disinfection.

### SUMMARY OF THE INVENTION

The invention is directed to a device and method for disinfecting a portion of a medical implement, such as a needleless hub or injection port, and protecting it when applied. The device comprises a shroud shaped to be engaged on the medical implement, with the shroud having an internal cavity. A disinfecting pad is located in the cavity, with the pad containing a liquid disinfectant. A covering is provided for maintaining the liquid disinfectant in the pad.

In accordance with one form of the invention, the shroud comprises a cap having internal threads in the cavity. The thread engage complementary threads of the medical implement when the cap is applied.

In one form of the invention, the covering comprises a seal applied to the shroud over the cavity. In this form of the invention, the seal can comprise a removable foil seal or a frangible foil seal. In another form of the invention, the covering comprises a pouch, with the shroud being sealed in the pouch. Preferably, the pouch is a foil pouch.

The pad is saturated with a liquid disinfectant of any appropriate nature. In accordance with the preferred form of the invention, the pad comprises a pair of pillows that are saturated with disinfectant when inserted in the internal cavity. The disinfectant can be any appropriate liquid, such as a chlorhexidine gluconate-based disinfectant.

### BRIEF DESCRIPTION OF THE DRAWINGS

The invention is described in greater detail in the following description of examples embodying the best mode of the invention, taken in conjunction with the drawing figures, in which:

FIG. 1 is a perspective view of one form of the invention,

FIG. 2 is a perspective view of the form of FIG. 1, but from the opposite side,

FIG. 3 is an elevational view thereof,

FIG. 4 is a top plan view of the invention as illustrated in FIG. 3,

FIG. 5 is a cross-sectional illustration of the invention as illustrated in FIG. 3,

FIG. 6 is an enlarged partial cross-sectional illustration of the form of the invention shown in FIG. 5,

FIG. 7 is an enlarged cross-sectional illustration of a second form of the invention,

FIG. 8 is a plan view of a pouch for containing the form of the invention shown in FIG. 7, and

FIG. 9 is an illustration of a typical needleless hub or port with which the invention is used.

### DESCRIPTION OF EXAMPLES EMBODYING THE BEST MODE OF THE INVENTION

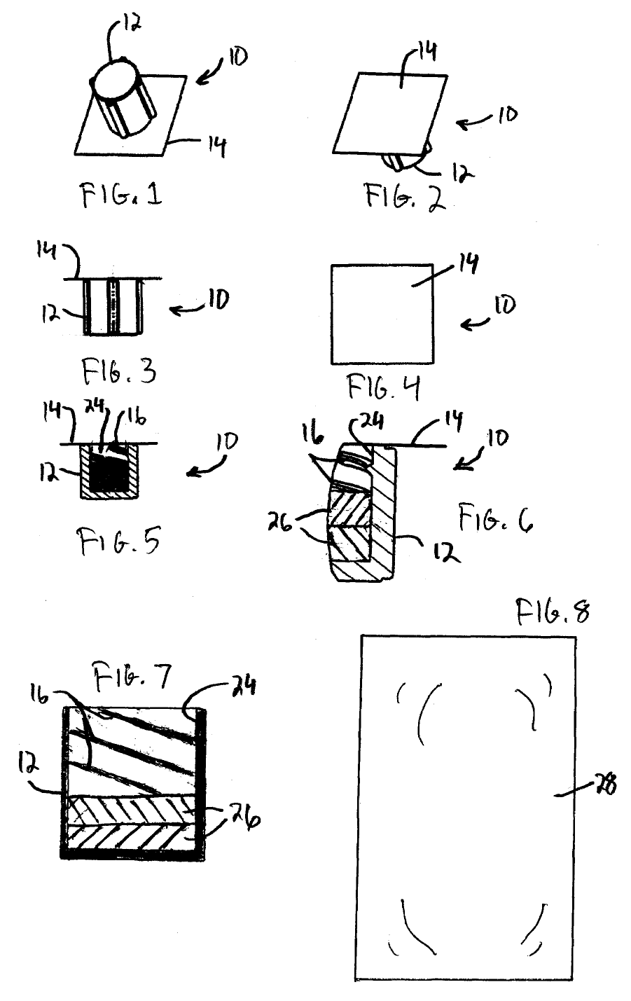

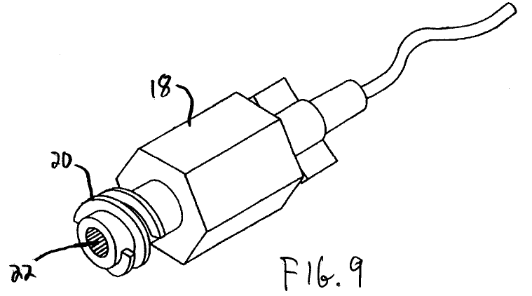

A first form of the device according to the invention is shown generally at 10 in FIGS. 1 through 6. The device 10 includes two primary components, a shroud in the form of a cap 12, and a covering in the form of a seal 14.

The cap 12 preferably is formed with internal threads 16 of the luer variety, which are formed to engage similar threads of a needleless hub or injection port, such as the hub 18 illustrated in FIG. 9, having threads 20 extending about a port 22. The hub 18 shown in FIG. 9 is conventional, and is therefore not described in greater detail. If the cap is to be applied to some other type of device, the threads 20 may or may not be present, as needed.

The cap 12 includes a disinfecting pad located in an inner cavity 24. While the pad can be of any form, preferably the pad is a pair of pillows 26, best illustrated in FIG. 6, which are saturated with an appropriate disinfectant solution, such as chlorhexidine gluconate (CHG). The pillows 26 can be of any material that absorbs liquid and is somewhat compressible so that when the cap 12 is applied as described below, the pillows 26 properly seat on the port 22 for disinfection purposes.

The seal 14 is preferably a foil seal that is applied to, and seals, the open end of the cavity 24. The means of application of the seal 14 to the cap 12 can be conventional, such as by use of adhesives, and is therefore not described in greater detail.

The seal 14 can be frangible or removeable, or both. The purpose of the seal 14 is to maintain the integrity of the disinfecting solution in the pillows 26 until such time as the device 10 is used.

A second form of the invention is shown in FIG. 7, and is actually preferably identical to the first form shown in FIGS. 1 through 6, except that the seal 14 is omitted. Thus, the same reference numerals in relation to the cap 12 of FIGS. 1 through 6 are also used in connection with the cap 12 of FIG. 7.

In this form of the invention, in order to maintain the integrity of the disinfecting solution in the pillows 26, a different form of covering is utilized. Instead of a seal 14, this form of the invention includes a pouch 28. The pouch 28 can be any conventional pouch which is fluid impervious, and is formed to contain the cap 12 therewithin. Preferably the pouch 28 is made of foil, which can either be separated to gain access to the cap 12 therewithin, or can be breached in some other manner to gain access to the cap 12, such as tearing or cutting.

In use, the covering in the form of the foil 14 or the pouch 28 is breached by removing or fracturing its structure, and the cap 12 is then applied to the hub 18 on the threads 20 so that the pad in the form of the pillows 26 engages the port 22. The disinfectant solution contained in the pillows 22 then disinfects the port 22, and with the cap in place on the hub 18, the port 22 remains covered and disinfected.

The invention has been described in relation to use with a luer end. However, as will be evident to one skilled in the art, the invention can be used in connection with all types of fittings, whether threaded or not, and whether needleless or needle-engaged. Various changes can be made to the invention without departing from the spirit thereof or scope of the following claims.

What is claimed is:

1. A device for disinfecting a portion of a medical implement, comprising,
a. a shroud shaped to be engaged on the medical implement, said shroud having an internal cavity,
b. a disinfecting pad located in said cavity, said pad containing a liquid disinfectant, and
c. a covering for maintaining the liquid disinfectant in said pad.

2. The device according to claim 1, in which said shroud comprises a cap having internal threads in said cavity.

3. The device according to claim 1, in which said covering comprises a seal applied to said shroud over said cavity.

4. The device according to claim 3, in which said seal comprises a removable foil seal.

5. The device according to claim 3, in which said seal comprises a frangible foil seal.

6. The device according to claim 1, in which said covering comprises a pouch for said shroud, said shroud being sealed in said pouch.

7. The device according to claim 6, in which said pouch is foil.

8. The device according to claim 1, in which said pad comprises a pair of saturated pillows.

9. A method of covering and disinfecting an external injection port, comprising the steps of:
a. providing a shroud for said port, said shroud having an internal cavity with a disinfecting pad located in said cavity and a covering for maintaining the liquid disinfectant in said paid, and
b. breaching said covering and applying said shroud to said port so that said pad engages and disinfects said port.

10. The method according to claim 4, in which said shroud comprises a cap having internal threads, and the applying step of method step “b” comprises screwing said cap onto said port.

11. The method according to claim 9, in which said covering comprises a foil seal, and the breaching step of method step “b” comprises removing said foil seal.

12. The method according to claim 9, in which said covering comprises a foil seal, and the breaching step of method step “b” comprises breaking said foil seal.

13. The method according to claim 9, in which said covering comprises a pouch, and the breaching step of method step “b” comprises opening said pouch.

14. The method according to claim 9, in which said covering comprises a foil pouch, and the breaching step of method step “b” comprises fracturing said foil.
